(19) United States
(12) Patent Application Publication
(10) Pub. No.: US 2008/0009048 A1
(43) Pub. Date: Jan. 10, 2008

(54) Liquefaction of Starch Containing Material

(75) Inventors: Swapnil BHARGAVA, Thousand Oaks, CA (US); Scott Dennis KOHL, Wichita, KS (US)

Correspondence Address: NOVOZYMES NORTH AMERICA, INC., 500 FIFTH AVENUE, SUITE 1600, NEW YORK, NY, 10110, US

(73) Assignees: Novozymes North America, Inc., Franklinton, NC (US); ICM Inc., Coiwich, KS (US)

(21) Appl. No.: 11/718,641

(22) PCT Filed: Nov. 4, 2005
(86) PCT No.: PCT/US05/40098

§ 371 Date: May 4, 2007

Related U.S. Application Data
(60) Provisional application No. 60/626,038, filed on Nov. 8, 2004.

(51) Int. Cl. C12P 7/06; C12P 19/02

(52) U.S. Cl. 435/161; 435/105

(57) ABSTRACT

The present invention relates to method of liquefying starch-containing material, wherein starch-containing material is subjected to a bacterial alpha-amylase at (a) a temperature around 50-80° C. for 10-180 minutes, followed by (b) treatment at a higher temperature than used in step (a) for 1-60 minutes.

### FIELD OF THE INVENTION

The present invention relates to improved methods of liquefying starch-containing material suitable as steps in processes for producing ethanol. The invention also relates to processes of producing ethanol comprising liquefying starch-containing starting material in accordance with the liquefaction method of the invention.

### BACKGROUND OF THE INVENTION

Liquefaction is a well known process step in the art of producing ethanol from starch-containing materials. During liquefaction starch is converted to shorter chains and less viscous dextrins. Generally liquefaction involves gelatinization of starch simultaneously with or followed by addition of alpha-amylase. Even though liquefaction methods suitable for ethanol production have been improved significantly over the last couple of decades there is still a need for improvements.

### SUMMARY OF THE INVENTION

The object of the present invention is to provide improved methods of liquefying starch-containing material suitable as a step in processes for producing ethanol. The invention also provides ethanol production processes including a liquefaction method of the invention.

According to the first aspect the invention relates to a method of liquefying starch-containing material, wherein said starch-containing material is subjected to a bacterial alpha-amylase at

(a) a temperature around 50-80° C. for 10-180 minutes, followed by

(b) treatment at a higher temperature than used in step (a) for 1-60 minutes.

According to the second aspect, the invention relates to a process of producing ethanol from starch-containing material by fermentation, said process comprises:

(i) liquefying starch-containing material in accordance with a liquefaction method of the invention;

(ii) saccharifying the liquefied mash obtained;

(iii) fermenting the material using a fermenting organism.

The term “mash” is used for liquefied starch-containing material, such as liquefied whole grains. In preferred embodiments the saccharification and fermentation in steps (ii) and (iii) are carried out as a simultaneous saccharification and fermentation process (SSF process).

A similar staging approach may be implemented. According to this embodiment the process of producing ethanol from starch-containing material by fermentation, comprises the following steps:

(i) liquefying starch-containing material with a bacterial alpha-amylase at a temperature around 50-80° C. for 10-180 minutes,

(ii) saccharifying the liquefied mash obtained in step (i);

(iii) fermenting the material using a fermenting organism;

(iv) recovering the ethanol obtained in step (iii);

(v) subjecting the ethanol stripped fermented material to a second liquefaction step using a bacterial alpha-amylase at a higher temperature than in step (i) for 1-60 minutes;

(vi) saccharifying the liquefied material obtained in step (v);

(vii) fermenting the material using a fermenting organism.

Optionally ethanol is recovery after fermentation step (vii). In preferred embodiments the saccharification and fermentation in step (ii) and (iii) and/or (vi) and (vii), respectively, are carried out as simultaneous saccharification and fermentation processes (SSF process).

### BRIEF DESCRIPTION OF THE DRAWINGS

FIG. 1: Analysis of ethanol yields for temperature modified liquefactions in SSF fermentation

FIG. 2: Amplified profiles for the initial fermentation span showing fermentation progress for different liquefaction treatments.

### DETAILED DESCRIPTION OF THE INVENTION

The present invention provides improved liquefaction processes suitable as steps in processes for producing ethanol. The invention also relates to a process of producing ethanol comprising a liquefaction method of the invention. The ethanol end-product may be used as, e.g., fuel ethanol; drinking ethanol, i.e., potable neutral spirits; or industrial ethanol.

Liquefaction

According to the present invention “liquefaction” is a process in which starch-containing material is broken down (hydrolyzed) into maltodextrins (dextrins). Because starch-containing material typically is heated to temperatures above the gelatinization temperature the liquefaction also helps the handling by thinning the starch-containing slurry. Liquefaction is usually carried out using a bacterial alpha-amylase at temperatures above 85° C. for about 90 minutes.

The inventors have now surprisingly found that when liquefying a starch-containing material in at least two stages, first at a temperature significantly lower than 85° C. for a suitable period of time and then at or around 85° C. for a period of time, advantages are obtained. For instance, the inventors have shown (see the Examples) that the fermentation rate and ethanol yield is higher when including a liquefaction method of the present invention in an ethanol production process compared to the corresponding process carried out at standard conditions.

Thus, according to the first aspect the invention relates to a method of liquefying starch-containing material, wherein starch-containing material is subjected to a bacterial alpha-amylase at

(a) a temperature around 50-80° C. for 10-180 minutes, followed by

(b) treatment at a higher temperature than used in step (a) for 1-60 minutes.

According to the second aspect the invention relates to a process of producing ethanol from starch-containing material by fermentation, said process comprises:

(i) liquefying said starch-containing material in accordance with the invention;

(ii) saccharifying the liquefied mash obtained;

(iii) fermenting the material using a fermenting organism.

In a preferred embodiment the alpha-amylase treatment in step (a) is carried out at a temperature between 65-75° C., preferably around 70° C. In a preferred embodiment the alpha-amylase treatment in step (a) is carried out for 30-150 minutes, preferably 60-120 minutes, especially around 90 minutes.

In a preferred embodiment the alpha-amylase treatment in step (b) is carried out at a temperature from above 80-110° C., preferably between 80 and 90° C., especially around 85° C., preferably for 5-40 minutes. The alpha-amylase treatment in step (b) is in a preferred embodiment carried out for 2-40 minutes, preferably around 5 minutes. Step (b) may be carried out as a jet-cooking step, preferably carried out at 90-110° C., preferably around 105° C., for 1-15 minutes, preferably for 3-10 minute, especially around 5 minutes. The pH during liquefaction is between 4.5-6.5, preferably between 5.2 and 6.2.

In a preferred embodiment the starch-containing material is milled corn, but other starch-containing materials are described in the “Starch-containing material”-section below.

The bacterial alpha-amylase may be any bacterial alpha-amylase, preferred a Bacillus alpha-amylases mentioned in the “Bacterial Alpha-Amylase”-section below.

Without being limited to any theory it is believed that exposing a corn slurry to a temperature significantly lower than the standard liquefaction temperature of 85° C. (i.e., closer to the gelatinization temperature) results in a configuration arrangement where only a part of the starch molecule is exposed to alpha-amylase. This results in smaller sugar cuts. Once smaller sugars are generated, raising the temperature above gelatinization temperature extends the remaining starch molecule and thereby exposing the complete chain to alpha-amylase. This results in generation of longer polysaccharides which are acted upon by glucoamylase during SSF for glucose generation. Portion of generated sugars which are smaller in lengths, helps in making fermentation faster since they are taken up by yeast directly while portion of longer chains act as suitable substrate for glucoamylase therefore making fermentation more efficient.

Starch-Containing Material

The starch-containing material used according to the present invention is selected from the group consisting of: tubers, roots and whole grain; and any combinations of the forgoing. In an embodiment, the starch-containing material is obtained from cereals. The starch-containing material may, e.g., be selected from the groups consisting of corns, cobs, wheat, barley, cassava, sorghum, rye, milo, and potatoes; or any combination of the forgoing.

If the liquefaction method of the invention is included in an ethanol process of the invention, the raw starch-containing material is preferably whole grain or at least mainly whole grains. A wide variety of starch-containing whole grain crops may be used as raw material including: corn (maize), milo, potato, cassava, sorghum, wheat, and barley. Thus, in one embodiment, the starch-containing material is whole grain selected from the group consisting of corn (maize), milo, potato, cassava, sorghum, wheat, and barley; or any combinations thereof. In a preferred embodiment, the starch-containing material is whole grain selected from the group consisting of corn, wheat, and barley; or any combinations thereof.

The raw material may also consist of or comprise a side-stream from starch processing, e.g., C₆ carbohydrate containing process streams, that are not suited for production of syrups.

Milling

In a preferred embodiment of the invention the starch-containing material is milled before step (a), i.e., before the primary liquefaction. Thus, in a particular embodiment, the liquefaction method further comprises, prior to the primary liquefaction step, i.e., prior to step (a), the steps of:

i. milling of starch-containing material, such as whole grains;

ii. forming a slurry comprising the milled starch-containing material and water.

The aqueous slurry contains from 10-40 wt-%, especially 25-35 wt-% starch-containing material. The starch-containing material, such as whole grains, is milled in order to open up the structure and allowing for further processing. Two processes of milling are normally used in ethanol production processes: wet and dry milling. The term “dry milling” denotes milling of the whole grain. In dry milling the whole kernel is milled and used in the remaining part of the process. Wet milling gives a good separation of germ and meal (starch granules and protein) and is with a few exceptions applied at locations where there is a parallel production of syrups. Dry milling is preferred in processes aiming at producing ethanol.

The term “grinding” is also understood as milling. In a preferred embodiment of the invention dry milling is used.

Ethanol Process

An ethanol production process of the invention generally involves the steps of liquefaction, saccharification, fermentation, and optionally recovering the ethanol product, preferably by distillation.

According to this aspect, the invention relates to a process of producing ethanol from starch-containing material by fermentation, said process comprises:

(i) liquefying starch-containing material in accordance with a liquefaction method of the invention;

(ii) saccharifying the liquefied mash obtained;

(iii) fermenting the material using a fermenting organism.

The term “mash” is used for liquefied starch-containing material, such as liquefied whole grains.

Saccharification

“Saccharification” is a step in which the maltodextrin (such as the product from liquefaction) is converted to low molecular sugars DP₁₋₃ (i.e., carbohydrate source) that can be metabolized by a fermenting organism, such as yeast. Saccharification is well known in the art and is typically performed enzymatically using one or more carbohydrate-source generating enzymes which are defined below in the “Carbohydrate-source generating enzyme”-section. The saccharification step comprised in a process for producing ethanol of the invention may be a well known saccharification step in the art. In one embodiment glucoamylase is used for treating the liquefied starch-containing material. A full saccharification step may last up to from 20 to 100 hours, preferably about 24 to about 72 hours, and is often carried out at temperatures from about 30 to 65° C., and at a pH between 4 and 6, normally around pH 4.5-5.0. However, it is often more preferred to do a pre-saccharification step, lasting for about 40 to 90 minutes, at temperature of between 30-65° C., typically about 60° C., followed by complete saccharification during fermentation in a simultaneous saccharification and fermentation process (SSF). The most widely used process in ethanol production is the simultaneous saccharification and fermentation (SSF) process, in which there is no holding stage for the saccharification, meaning that fermenting organism(s), such as yeast, and enzyme(s) is(are) added together. In SSF processes, it is common to introduce a pre-saccharification step at a temperature between about 40 and 60° C., preferably around 50° C., just prior to the fermentation. In preferred embodiments the saccharification and fermentation in step (ii) and (iii) are carried out as a simultaneous saccharification and fermentation process (SSF process).

Fermentation

In an ethanol production process of the invention the “fermenting organism” is applied to the saccharified material. The term “fermenting organism” refers to any organism suitable for use in a desired fermentation process. Suitable fermenting organisms are according to the invention capable of fermenting, i.e., converting sugars, such as glucose and/or maltose, directly or indirectly into ethanol. Examples of fermenting organisms include fungal organisms, such as yeast. Preferred yeast includes strains of Saccharomyces, in particular Saccharomyces cerevisiae. Commercially available yeast includes, e.g., RED STAR®/Lesaffre Ethanol Red™ (available from Red Star/Lesaffre, USA), SUPERSTART™ (available from Alltech), GERT STRAND (available from Gert Strand AB, Sweden) and FERMIOL™ (available from DSM Specialties). In preferred embodiments, yeast is applied to the saccharified mash. Fermentation is ongoing for 24-96 hours, such as typically 35-65 hours. In preferred embodiments, the temperature is generally between about 26-34° C., in particular about 32° C., and the pH is generally from pH 3-6, preferably around pH 4-5. Yeast cells are preferably applied in amounts of 10⁵ to 10¹², preferably from 10⁷ to 10¹⁰, especially 5×10⁷ viable yeast count per ml of fermentation broth. During the ethanol producing phase the yeast cell count should preferably be in the range from 10⁷ to 10¹⁰, especially around 2×10⁸. Further guidance with regards to using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference.

Recovery of Ethanol

Optionally the ethanol is recovery after fermentation, preferably by including a step of

(iv) distillation to obtain the ethanol;

wherein the fermentation in step (iii) and the distillation in step (iv) is carried out simultaneously or separately/sequential; optionally followed by one or more process steps for further refinement of the ethanol.

Alternative Staging Liquefaction Process

The inventors have found that a similar liquefaction staging approach can be implemented. According to this approach, liquefaction is initially done with lower/similar amounts (e.g., 1 to ⅔ of the total amount) of alpha-amylase at a temperature significantly below 85° C. for a suitable period of time and then subjected to SSF. After fermentation, ethanol is stripped off from the fermented material. The stripped fermented material is then subjected to liquefaction at around 85° C. for a suitable period of time. Once the second liquefaction step is completed, the mash can again be subjected to SSF. Since there will be lower ethanol concentration to begin with in secondary SSF, no ethanol inhibition will be seen, potentially resulting in overall better ethanol yield.

Thus, according to this staging approach the invention relates to a process of producing ethanol from starch-containing material by fermentation, comprises the following steps:

(i) liquefying starch-containing material with a bacterial alpha-amylase at a temperature around 50-80° C. for 10-180 minutes,

(ii) saccharifying the liquefied mash obtained in step (i);

(iii) fermenting the material using a fermenting organism;

(iv) recovering the ethanol obtained in step (iii);

(v) subjecting the ethanol stripped fermented material to a second liquefaction step using a bacterial alpha-amylase at a significantly higher temperature than in step (i) for 1-60 minutes;

(vi) saccharifying the liquefied material obtained in step (v);

(vii) fermenting the material using a fermenting organism.

Optionally ethanol is recovery after fermentation step (vii). In preferred embodiments the saccharification and fermentation in step (ii) and (iii) and/or (vi) and (vii), respectively, are carried out as simultaneous saccharification and fermentation processes (SSF process).

In an embodiment saccharification and fermentation are carried out as a simultaneous saccharification and fermentation process (SSF process). In a preferred embodiment of the invention starch-containing raw material, such as whole grains, preferably corn, is dry milled in order to open up the structure and allow for further processing.

### ENZYMES

Bacterial Alpha-Amylase

According to the invention the bacterial alpha-amylase is preferably derived from the genus Bacillus. In a preferred embodiment the Bacillus alpha-amylase is derived from a strain of B. licheniformis, B. amyloliquefaciens, B. subtilis or B. stearothermophilus, but may also be derived from other Bacillus sp. Specific examples of contemplated alpha-amylases include the Bacillus licheniformis alpha-amylase shown in SEQ ID NO: 4, the Bacillus amyloliquefaciens alpha-amylase SEQ ID NO: 5 and the Bacillus stearothermophilus alpha-amylase shown in SEQ ID NO: 3 in WO 99/19467 (all sequences hereby incorporated by reference). In an embodiment of the invention the alpha-amylase may be an enzyme having a degree of identity of at least 60%, preferably at least 70%, more preferred at least 80%, even more preferred at least 90%, such as at least 95%, at least 96%, at least 97%, at least 98%, or at least 99% identity to any of the sequences shown in SEQ ID NOS: 1, 2, 3, 4 or 5, respectively, in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,187,576, 6,297,038, and 6,867,031 (hereby incorporated by reference) and include Bacillus stearothermophilus alpha-amylase (BSG alpha-amylase) variants having a double deletion corresponding to delta(181-182) compared to the wild-type BSG alpha-amylase amino acid sequence set forth in SEQ ID NO:3 disclosed in WO 99/19467 (hereby incorporated by reference). Preferred are also Bacillus alpha-amylases, especially Bacillus stearothermophilus alpha-amylases, which have a single or double deletion in positions or corresponding to positions 181-182 in the BSG alpha-amylase. Even more preferred are Bacillus alpha-amylases having a single or double deletion in positions or corresponding to positions 181-182 in the BSG alpha-amylase that further comprises a N193F substitution (also denoted I181*+G182*+N193F) compared to the wild-type BSG alpha-amylase amino acid sequence set forth in SEQ ID NO:3 disclosed in WO 99/19467.

A hybrid alpha-amylase specifically contemplated comprises 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with one or more, especially all, of the following substitution: G48A+T49I+G107A+H156Y+A181T+N190F+I201F+A209V+Q264S (using the Bacillus licheniformis numbering in SEQ ID NO: 4 of WO 99/19467). Preferred are also variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467).

The bacterial alpha-amylase may be added in amounts well-known in the art. When measured in KNU units (described below in the “Materials & Methods”-section) the alpha-amylase activity is preferably present in between 0.5-5,000 NU/g of DS, in an amount of 1-500 NU/g of DS, or more preferably in an amount of 5-1,000 NU/g of DS, such as 10-100 NU/g DS.

Carbohydrate-Source Generating Enzyme

The term “carbohydrate-source generating enzyme” includes glucoamylase (being glucose generators), beta-amylase and maltogenic amylase (being maltose generators). A carbohydrate-source generating enzyme is capable of providing energy to the fermenting organism(s) used in a process of the invention for producing ethanol. The carbohydrate-source generating enzyme may be mixtures of enzymes falling within the definition. Especially contemplated mixtures are mixtures of at least a glucoamylase and an alpha-amylase, especially an acid alpha-amylase, even more preferred an acid fungal alpha-amylase. The ratio between acidic fungal alpha-amylase activity (AFAU) per glucoamylase activity (AGU) (AFAU per AGU) may in an embodiment of the invention be at least 0.1, in particular at least 0.16, such as in the range from 0.12 to 0.50.

Examples of contemplated glucoamylases, maltogenic amylases, and beta-amylases are set forth in the sections below.

Glucoamylase

A glucoamylase used according to the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al. (1984), EMBO J. 3 (5), p. 1097-1102), or variants thereof, such as disclosed in WO 92/00381, WO 00/04136 add WO 01/04273 (from Novozymes, Denmark); the A. awamori glucoamylase (WO 84/02921), A. oryzae (Agric. Biol. Chem. (1991), 55 (4), 941-949), or variants or fragments thereof.

Other Aspergillus glucoamylase variants include variants to enhance the thermal stability: G137A and G139A (Chen et al., 1996, Prot. Eng. 9: 499-505); D257E and D293E/Q (Chen et al., 1995, Prot. Engng. 8: 575-582); N182 (Chen et al., 1994, Biochem. J. 301: 275-281); disulphide bonds, A246C (Fierobe et al., 1996, Biochemistry, 35: 8698-8704; and introduction of Pro residues in position A435 and S436 (Li et al., 1997, Protein Engng. 10: 1199-1204. Other glucoamylases include Athelia rolfsii (previously denoted Corticium rolfsii) glucoamylase (see U.S. Pat. No. 4,727,026 and (Nagasaka, Y. et al. (1998) Purification and properties of the raw-starch-degrading glucoamylases from Corticium rolfsii, Appl Microbiol Biotechnol 50:323-330), Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS, preferably 0.1-10 AGU/g DS, especially between 1-5 AGU/g DS, such as 0.5 AGU/g DS.

Beta-Amylase

At least according to the invention the a beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, 1979, Progress in Industrial Microbiology, 15: 112-115). These beta-amylases are characterized by having optimum temperatures in the range from 40° C. to 65° C. and optimum pH in the range from 4.5 to 7. A commercially available beta-amylase from barley is NOVOZYM™ WBA from Novozymes A/S, Denmark and SPEZYME™ BBA 1500 from Genencor Int., USA.

Maltogenic Amylase

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic alpha-amylase from Bacillus stearothermophilus strain NCIB 11837 is commercially available from Novozymes A/S under the tradename MALTOGENASE™. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram DS or 0.05-5 MANU/g DS.

Production of Enzymes

The enzymes referenced herein may be derived or obtained from any suitable origin, especially bacterial, fungal, and yeast origin. The term “derived” or means in this context that the enzyme may have been isolated from an organism where it is present natively, i.e., the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e., a recombinantly produced enzyme which is a mutant and/or a fragment of a native amino acid sequence or an enzyme produced by nucleic acid shuffling processes known in the art. Within the meaning of a native enzyme are included natural variants. Furthermore, the term “derived” includes enzymes produced synthetically by, e.g., peptide synthesis. The term “derived” also encompasses enzymes which have been modified e.g., by glycosylation, phosphorylation, or by other chemical modification, whether in vivo or in vitro. The term “obtained” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by, e.g., peptide synthesis. With respect to recombinantly produced enzymes the terms “obtained” and “derived” refers to the identity of the enzyme and not the identity of the host organism in which it is produced recombinantly.

The enzymes may also be purified. The term “purified” as used herein covers enzymes free from other components from the organism from which it is derived. The term “purified” also covers enzymes free from components from the native organism from which it is obtained. The enzymes may be purified, with only minor amounts of other proteins being present. The expression “other proteins” relate in particular to other enzymes. The term “purified” as used herein also refers to removal of other components, particularly other proteins and most particularly other enzymes present in the cell of origin of the enzyme of the invention. The enzyme may be “substantially pure,” that is, free from other components from the organism in which it is produced, that is, for example, a host organism for recombinantly produced enzymes. In preferred embodiment, the enzymes are at least 75% (w/w) pure, more preferably at least 80%, at least 85%, at least 90%, at least 95%, at least 96%, at least 97%, at least 98%, or at least 99% pure. In another preferred embodiment, the enzyme is 100% pure.

The enzymes used according to the present invention may be in any form suitable for use in the processes described herein, such as, e.g., in the form of a dry powder or granulate, a non-dusting granulate, a liquid, a stabilized liquid, or a protected enzyme. Granulates may be produced, e.g., as disclosed in U.S. Pat. Nos. 4,106,991 and 4,661,452, and may optionally be coated by process known in the art. Liquid enzyme preparations may, for instance, be stabilized by adding stabilizers such as a sugar, a sugar alcohol or another polyol, lactic acid or another organic acid according to established process. Protected enzymes may be prepared according to the process disclosed in EP 238,216.

Even if not specifically mentioned in context of a method or process of the invention, it is to be understood that the enzyme(s) or agent(s) is(are) used in an “effective amount”.

### MATERIAL & METHODS

Enzymes:

Bacterial Alpha-Amylase A (BAAA): Bacillus stearothermophilus alpha-amylase variant with the mutations: I181*+G182*+N193F disclosed in U.S. Pat. No. 6,187,576 and available on request from Novozymes A/S, Denmark.
Glucoamylase T; Glucoamylase derived from Talaromyces emersonii and disclosed as SEQ ID NO: 7 in WO 99/28448 available on request from Novozymes A/S, Denmark.
Yeast: RED STAR™ available from Red Star/Lesaffre, USA
   Methods:
   Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.±0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

Determination of FAU Activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| | |
|---|---|
| Substrate | Soluble starch |
| Temperature | 37° C. |
| pH | 4.7 |
| Reaction time | 7-20 minutes |

Determination of Acid Alpha-Amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, Denmark, glucoamylase wild-type Aspergillus niger G1, also disclosed in Boel et al. (1984), EMBO J. 3 (5), p. 1097-1102) and WO 92/00381). The neutral alpha-amylase in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of color is therefore directly proportional to the concentration of starch. Amylase activity is determined using reverse colorimetry as a reduction in the concentration of starch under specified analytic conditions.

$$\begin{matrix} & Alpha \text{-} amylase & \\ Starch + Iodine & -> & Dextrins + Oligosaccharides \\ & 40 ° C . , pH 2.5 & \\ Blue \text{/} Violet & t = 23 sec . & Decoloration\end{matrix}$$

Standard Conditions/Reaction Conditions: (Per Minute)

| | |
|---|---|
| Substrate: | Starch, approx. 0.17 g/L |
| Buffer: | Citate, approx. 0.03 M |
| Iodine (I₂): | 0.03 g/L |
| CaCl_{2:} | 1.85 mM |
| pH: | 2.50 ± 0.05 |
| Incubation temperature: | 40° C. |
| Reaction time: | 23 seconds |
| Wavelength: | lambda = 590 nm |
| Enzyme concentration: | 0.025 AFAU/mL |
| Enzyme working range: | 0.01-0.04 AFAU/mL |

If further details are preferred these can be found in EB-SM-0259.02/01 available on request from Novozymes A/S, Denmark, and incorporated by reference.

Acid Alpha-Amylase Units (AAU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

Standard Conditions/Reaction Conditions:

| | |
|---|---|
| Substrate: | Soluble starch. Concentration approx. |
| | 20 g DS/L. |
| Buffer: | Citrate, approx. 0.13 M, pH = 4.2 |
| Iodine solution: | 40.176 g potassium iodide + 0.088 g iodine/L |
| City water | 15°-20° dH (German degree hardness) |
| pH: | 4.2 |
| Incubation temperature: | 30° C. |
| Reaction time: | 11 minutes |
| Wavelength: | 620 nm |
| Enzyme concentration: | 0.13-0.19 AAU/mL |
| Enzyme working range: | 0.13-0.19 AAU/mL |

The starch should be Lintner starch, which is a thin-boiling starch used in the laboratory as colorimetric indicator. Lintner starch is obtained by dilute hydrochloric acid treatment of native starch so that it retains the ability to color blue with iodine. Further details can be found in EP0140410B2, which disclosure is hereby included by reference.

Glucoamylase Activity (AGI)

Glucoamylase (equivalent to amyloglucosidase) converts starch into glucose. The amount of glucose is determined here by the glucose oxidase method for the activity determination. The method described in the section 76-11 Starch—Glucoamylase Method with Subsequent Measurement of Glucose with Glucose Oxidase in “Approved methods of the American Association of Cereal Chemists”. Vol.1-2 AACC, from American Association of Cereal Chemists, (2000); ISBN: 1-891127-12-8.

One glucoamylase unit (AGI) is the quantity of enzyme which will form 1 micromol of glucose per minute under the standard conditions of the method.

Standard Conditions/Reaction Conditions:

| | |
|---|---|
| Substrate: | Soluble starch. |
| | Concentration approx. 16 g dry matter/L. |
| Buffer: | Acetate, approx. 0.04 M, pH = 4.3 |
| pH: | 4.3 |
| Incubation temperature: | 60° C. |
| Reaction time: | 15 minutes |
| Termination of the reaction: | NaOH to a concentration of approximately |
| | 0.2 g/L (pH˜9) |
| Enzyme concentration: | 0.15-0.55 AAU/mL |

The starch should be Lintner starch, which is a thin-boiling starch used in the laboratory as colorimetric indicator. Lintner starch is obtained by dilute hydrochloric acid treatment of native starch so that it retains the ability to color blue with iodine.

Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| | |
|---|---|
| AMG incubation: | |
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |
| Color reaction: | |
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

Determination of Maltogenic Amylase Activity (MANU)

One MANU (Maltogenic Amylase Novo Unit) may be defined as the amount of enzyme required to release one micro mole of maltose per minute at a concentration of 10 mg of maltotriose (Sigma M 8378) substrate per ml of 0.1 M citrate buffer, pH 5.0 at 37° C. for 30 minutes.

Identity Between Two Sequences

The degree of identity between two amino acid sequences is determined by the Clustal method (Higgins, 1989, CABIOS 5:151-153) using the LASERGENE™ MEGALIGN™ software (DNASTAR, Inc., Madison, Wis.) with an identity table and the following multiple alignment parameters: Gap penalty of 10, and gap length penalty of 10. Pairwise alignment parameters were Ktuple=1, gap penalty=3, windows=5, and diagonals=5].

### EXAMPLES

### Example 1

### Ethanol Yields from Temperature Staging Liquefactions

To investigate the effect of temperature staging on liquefaction process, 5 different conditions for liquefaction were tested as listed in Table 1. To begin with milled corn was used to make 32% slurry with tap water and backset (thin stillage) (1:1). The backset used in the mixing had 7.25% solids. The pH in all the liquefactions was adjusted to 5.8 using diluted H₂SO₄. Once pH was adjusted, constant dose (0.04% w/w) of Bacterial Alpha-Amylase A (BAAA) was added in the corn mash. The slurry was then subjected to different temperature conditions for primary and secondary liquefaction as shown in Table 1. Once the liquefaction was complete, liquefied material was subjected to simultaneous saccharification and fermentation (SSF) after cooling down to room temperature.

TABLE 1
Liquefaction conditions for Temperature study

| Liquefaction | Time (minutes) at 85° C. | Time (minutes) at 70° C. |
|---|---|---|
| 1 | 0 | 90 |
| 2 | 5 | 85 |
| 3 | 10 | 80 |
| 4 | 20 | 70 |
| 5 | 30 | 60 |

The effect of liquefaction treatment on SSF was evaluated via mini-scale fermentations. Approximately 4 g of mash was added to 16 mL polystyrene tubes. Tubes were then dosed with Glucoamylase T for saccharification (0.05% w/w). After dosing the tubes with enzyme, they were inoculated with 0.04 mL/g mash of yeast propagate (RED STAR™) that had been grown for 21 hours on corn mash. Vials were capped with a screw on lid which had been punctured with a very small needle to allow gas release and vortexed briefly before weighing and incubation at 32° C. Fermentation progress was followed by weighing the tubes over time. Tubes were vortexed briefly before each weighing. Weight loss values were converted to ethanol yield (g ethanol/g DS) by the following formula:

$$g ethanol / g DS = \frac{g {CO}_{2} weight loss ⨯ \frac{1 mol {CO}_{2}}{44.0098 g {CO}_{2}} ⨯ \frac{1 mol ethanol}{1 mol {CO}_{2}} ⨯ \frac{46.094 g ethanol}{1 mol ethanol}}{g corn in tube ⨯ \% DS of corn}$$

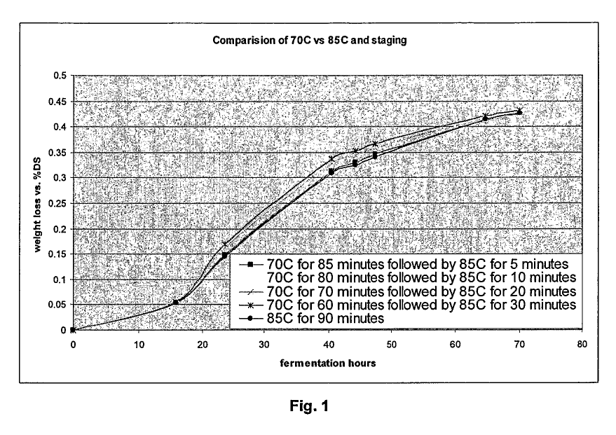

Ethanol yields from fermentation for the temperature staging study were analyzed by weight loss due to CO₂ release. The result is shown in FIG. 1.

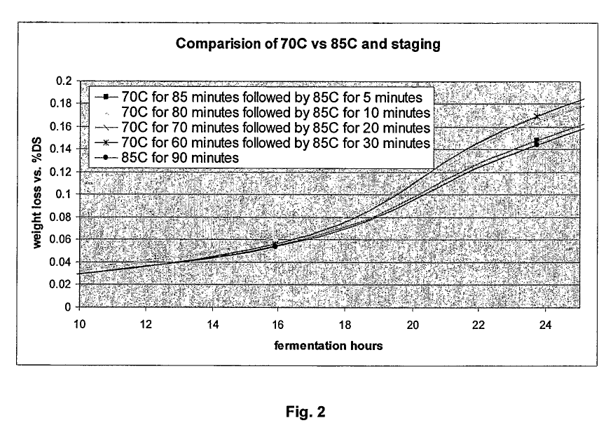

FIG. 2 shows this phenomenon more clearly in an amplified picture of fermentation curves. A significant color difference between liquefied mash obtained from a traditional liquefaction process and a liquefaction process of the invention was observed. A traditional liquefaction process where liquefaction is done at 85° C. over a time period of 1.5-2.0 hours showed dark brown color whereas liquefactions coupled with the 70° C. process showed more whitish color. This indicates that down stream processing in the staged liquefactions could potentially result in better quality of DDG (Distiller's Dried Grains).

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

1-46. (canceled)

47. A method of liquefying starch-containing material, comprising
(a) subjecting starch-containing material to a bacterial alpha-amylase at a temperature around 50-80° C. for 10-180 minutes, followed by
(b) treatment at a higher temperature than used in step (a) for 1-60 minutes.

48. The method of claim 47, wherein the starch-containing material is selected from the group consisting of corn, cob, wheat, barley, rye, milo and potatoes; or any combination of these.

49. The method of claim 47, wherein the method comprises a step of milling the starch-containing material before step (a).

50. The method of claim 47, wherein the starch-containing material is obtainable by a process comprising milling of whole grains.

51. The method of claim 47, wherein the alpha-amylase treatment in step (a) is carried out at a temperature in the range between 65-75° C.

52. The method of claim 47, wherein the alpha-amylase treatment in step (a) is carried out for 30-150 minutes.

53. The method of claim 47, wherein the alpha-amylase treatment in step (b) is carried out at a temperature in the range from 80-110° C.

54. The method of claim 47, wherein the alpha-amylase treatment in step (b) is carried out for 2-40 minutes.

55. The method of claim 48, wherein step (b) is carried out as a jet-cooking step.

56. The method of claim 47, wherein the pH during liquefaction is between 4.5-6.5.

57. The method of claim 47, further comprising prior to step (a) the steps of:
i) milling of starch-containing material;
ii) forming a slurry comprising the milled material and water.

58. The method of claim 57, wherein the milling step is a dry milling step.

59. The method of claim 57, wherein the milling step is a wet milling step.

60. The method of claim 47, wherein the starch-containing material is a side stream from starch processing.

61. The method of claim 47, wherein the bacterial alpha-amylase in step (a) is a Bacillus alpha-amylase.

62. A process of producing ethanol from starch-containing material, comprising:
(i) liquefying said starch-containing material as defined in claim 47;
(ii) saccharifying the liquefied mash obtained from step (i); and
(iii) fermenting the material using a fermenting organism.

63. The process of claim 62, further comprising recovery of the ethanol.

64. The process of claim 62, wherein the saccharification and fermentation is carried out as a simultaneous saccharification and fermentation process (SSF process).

65. The process of claim 64, wherein the saccharification or SSF process is carried out in the presence of a carbohydrate-source generating enzyme.

66. The process of claim 65, wherein the carbohydrate-source generating enzyme is a glucoamylase.

67. The process of claim 62, further comprising
(iv) distillation to obtain the ethanol;
wherein the fermentation in step (iii) and the distillation in step (iv) are carried out simultaneously or separately/sequential; optionally followed by one or more process steps for further refinement of the ethanol.

68. A process of producing ethanol from starch-containing material by fermentation, said process comprises:
(a) liquefying starch-containing material with a bacterial alpha-amylase at a temperature around 50-80° C. for 10-180 minutes,
(b) saccharifying the liquefied mash obtained in step (a);
(c) fermenting the material using a fermenting organism,
(d) recovering the ethanol obtained in step (c);
(e) subjecting the ethanol stripped fermented material to a second liquefaction step using a bacterial alpha-amylase at a higher temperature than in step (a) for 1-60 minutes;
(f) saccharifying the liquefied material obtained in step (e); and
(g) fermenting the material using a fermenting organism.
